(19) United States
(12) Patent Application Publication
(10) Pub. No.: US 2025/0177426 A1
(43) Pub. Date: Jun. 5, 2025

(54) METHODS AND COMPOSITIONS FOR TREATING DEMENTIA

(71) Applicant: Cognitive Synergy Corporation, San Diego, CA (US)

(72) Inventors: Asha Amritraj, San Diego, CA (US); Arnav Dhar, San Diego, CA (US)

(73) Assignee: Cognitive Synergy Corporation, San Diego, CA (US)

(21) Appl. No.: 18/966,092

(22) Filed: Dec. 2, 2024

Related U.S. Application Data
(60) Provisional application No. 63/604,390, filed on Nov. 30, 2023.

(51) Int. Cl. A61K 31/7048; A61K 31/7034

(52) U.S. Cl. CPC A61K 31/7048; A61K 31/7034

(57) ABSTRACT

Prodrugs are disclosed for treatment of cognitive disease such as Alzheimer's disease. A method for treatment includes administering a prodrug configured to deliver sinapic acid O-glucoside to a patient. Another method for treatment includes administering a prodrug configured to deliver neoliquiritin to a patient.

This application claims the benefit of U.S. Provisional Patent Application No. 63/604,390, entitled as “METHODS AND COMPOSITIONS FOR TREATING DEMENTIA”, filed Nov. 30, 2023, which is incorporated by reference in its entirety.

### FIELD OF THE INVENTION

This disclosure relates to compositions for treatment of cognitive disorders such as Alzheimer's disease.

### BACKGROUND

Alzheimer's disease is a common form of dementia. Although it may begin earlier in some cases, symptoms of Alzheimer's disease generally begin in individuals 65 years and older. The disease is characterized by a slow and gradual cognitive decline that manifests early as simple memory loss. Later, the individual gradually loses cognitive function to the point that they are entirely dependent on caregivers. In late-stage Alzheimer's, the individual often loses their ability to speak.

Despite widespread and omnipresent suffering from Alzheimer's disease, there is no cure. Indeed, there are only a handful of therapies to treat Alzheimer's disease, and they only offer marginal relief. And this is all while the cost of Alzheimer's disease in money, time, and anguish takes an increasing toll on the world population as life expectancy increases.

Although the cause of Alzheimer's disease is not completely understood, there is increasing evidence that Aβ protein (abeta) and tau protein play a significant role. For instance, Alzheimer's disease is characterized by the deposition of Aβ amyloid fibrils, which are extracellular deposits of Aβ protein plaques in the gray matter of the brain. Alzheimer's disease is further characterized by the deposition of neurofibrillary tangles, which are abnormal accumulations of tau protein inside neurons.

### SUMMARY

Methods and compositions for treatment of dementia are provided. An exemplary embodiment is a method for treatment of the patient. The method comprises administering an

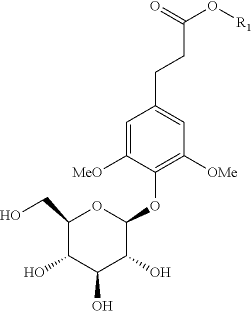

effective dose of a compound having the formula wherein R₁ includes

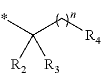

where n is an integer selected from 0 to 5, R₂, R₃, and R₄ are independently selected from the group consisting of H, C₁-C₆ alkyl, C₃-C₆ cycloalkyl, C₃-C₆ heterocycloalkyl, Ar, hAr, where Ar includes phenyl independently substituted with 0-5 substituents where each substituent is independently C₁-C₆ alkyl, C₃-C₆ cycloalkyl, Ar, hAr, where hAr includes a 5-7 membered heteroaryl ring independently substituted with 0-6 substituents comprising C₁-6 Alk containing 0-3 heteroatoms, Ar, hAr substituted with (Alk, OAlk, NHAlk, N(Alk)₂, or a halogen). R₁ may further include

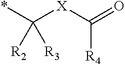

where X comprises O, NR, or bond, wherein R₂ and R₃ independently comprise H, C₁₋₆ Alk, C₃₋₆ cycloalkyl, or cyclized to form ring (each containing or substituted with 0-3 heteroatoms), Ar, or hAr, wherein R₄ is selected from the group consisting of C₁₋₆ Alk, C₃₋₆ cycloalkyl (each containing or substituted with 0-3 heteroatoms, NHAlk, N(Alk)₂, Ar, or hAr, wherein Ar comprises phenyl independently substituted with 0-5 substituents wherein each substituent is independently C₁-C₆ alkyl, C₃-C₆ cycloalkyl, Ar, hAr, wherein hAr comprises a 5-7 membered heteroaryl ring independently substituted with 0-6 substituents comprising C₁₋₆ Alk containing 0-3 heteroatoms, Ar, or hAr substituted with Alk, OAlk, NHAlk, N(Alk)₂, or a halogen. R₁ may further include

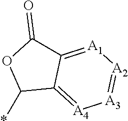

where A₁, A₂, A₃, and A₄ are independently selected from the group consisting of N or CR₂, where R₂ is selected from the group consisting of H, C₁₋₆ Alk, C₃₋₆ cycloalkyl, Ar, or hAr, where Ar comprises phenyl independently substituted with 0-5 substituents where each substituent is independently C₁-C₆ alkyl, C₃-C₆ cycloalkyl, Ar, hAr, where hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include

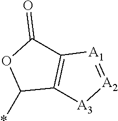

where A₁, A₂, and A₃ are independently selected from the group consisting of N, O, S, or CR2, where R₂ is selected from the group consisting of H, C₁₋₆ Alk, C₃₋₆ cycloalkyl, Ar, or hAr, wherein Ar comprises phenyl independently substituted with 0-5 substituents wherein each substituent is independently C₁-C₆ alkyl, C₃-C₆ cycloalkyl, Ar, or hAr, where hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include

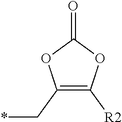

where R₂ is selected from the group consisting of H, C₁₋₆ Alk, C₃₋₆ cycloalkyl comprising or substituted with 0-3 heteroatoms, Ar, or hAr, wherein Ar comprises phenyl independently substituted with 0-5 substituents where each substituent is independently C₁-C₆ alkyl, C₃-C₆ cycloalkyl, Ar, hAr(alkyl, O-alkyl, NH-alkyl, N(alkyl)₂, wherein hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include

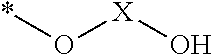

where X is selected from the group consisting of C₂₋₆ Alk, C₃₋₆ cycloalkyl each containing or substituted with 0-3 heteroatoms, Ar, hAr, or formula I with structure

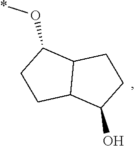 [SMILES: *O[C@H]1CCC2C1CC[C@H]2O]

where Ar includes phenyl independently substituted with 0-5 substituents wherein each substituent is independently C₁-C₆ alkyl, C₃-C₆ cycloalkyl, Ar, hAr(alkyl, O-alkyl, NH-alkyl, N(alkyl)₂, where hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. The term Alk may refer to an alkyl group. The effective dose may have a concentration of between about 10 μM and about 150 μM. The effective dose may have a concentration of between about 50 μM and about 100 μM. The effective dose may have a concentration of between about 100 μM and about 150 μM. The effective dose may have a concentration of between about 20 μM and about 40 μM. The effective dose may have a concentration of between about 40 μM and about 60 μM. The effective dose may have a concentration of between about 60 μM and about 80 μM. The effective dose may have a concentration of between about 80 μM and about 100 μM. The effective dose may have a concentration of between about 100 μM and about 120 μM. The effective dose may have a concentration of between about 100 μM and about 140 μM.

Another general aspect is a method for treatment of a patient. The method includes administering an effective dose of a compound having the formula

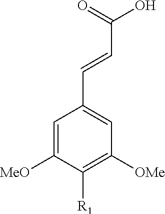

where R₁ is selected from the group consisting of

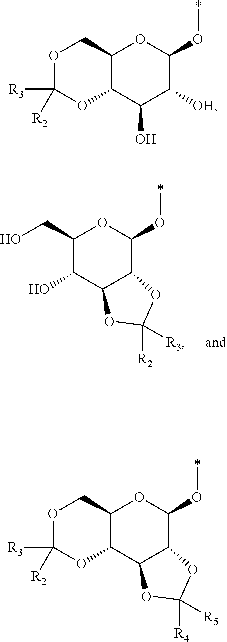

where R₂-R₅ are independently selected from the group consisting of H, C₁₋₆ Alk, C₃₋₆ cycloalkyl, Ar, hAr. In the alternative, R1 may include

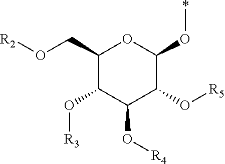

where R₂₋₅ are independently selected from the group consisting of H, C₁₋₆Alk, C₃₋₆ cycloalkyl, Ar, hAr, or group selected from

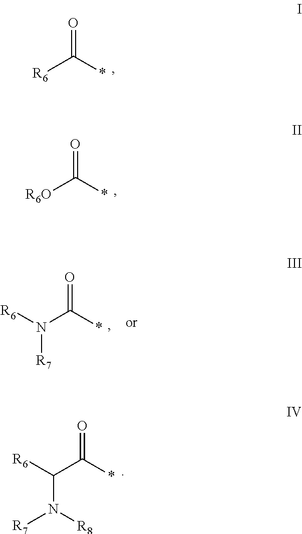

where R₂₋₅ are independently selected from the group consisting of H, C₁₋₆ Alk, C₃₋₆ cycloalk, Ar, hAr, I, II, III, or IV. R₆₋₈ may be independently selected from the group consisting of H, C₁₋₆ Alk, C₃₋₆ cycloalk, Ar, or hAr, wherein Ar comprises phenyl independently substituted with 0-5 substituents wherein each substituent is independently C₁-C₆ alkyl, C₃-C₆ cycloalkyl, Ar, hAr, wherein hAr comprises a 5-7 membered heteroaryl ring independently substituted with 0-6 substituents comprising _{C1-6} Alk containing 0-3 heteroatoms, Ar, hAr substituted with Alk, OAlk, NHAlk, N(Alk)₂, or a halogen. The effective dose may have a concentration of between about 20 μM and about 40 μM. The effective dose may have a concentration of between about 40 μM and about 60 μM. The effective dose may have a concentration of between about 60 μM and about 80 μM. The effective dose may have a concentration of between about 80 μM and about 100 μM. The effective dose may have a concentration of between about 100 μM and about 120 μM. The effective dose may have a concentration of between about 100 μM and about 140 μM.

An exemplary embodiment is a method for treatment of the patient. The method comprises administering an effective dose of a compound having the formula

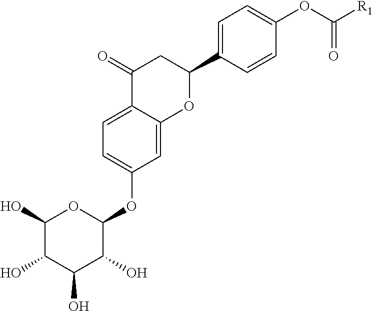

where R₁ includes the group

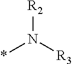

where R₂ and R₃ are independently selected from the group consisting of: H, C₁-C₆ alkyl, C₃-C₆ cycloalkyl, or cyclized to form a ring wherein each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include the group

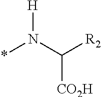

where R₂ is independently selected from the group consisting of: H, C₁-C₆ alkyl, C₃-C₆ cycloalkyl, or cyclized to form a ring wherein each ring contains or is substituted with 0-3 heteroatoms, Ar, hAr, where Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include the group

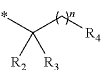

where n is an integer selected from 0 to 5, wherein R2, R3, and R4 are independently selected from the group consisting of: H, C₁-C₆ alkyl, C₃-C₆ cycloalkyl, or cyclized to form a ring wherein each ring contains or is substituted with 0-3 heteroatoms, Ar, hAr, where Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ Alk containing 0-3 heteroatoms, Ar, or hAr substituted with Alk, OAlk, NHAlk, N(Alk)₂, or a halogen. R1 may further include the group

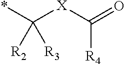

where X is selected from the group consisting of O, NR, or a bond, R₂ and R₃ are independently selected from the group consisting of: H, C₁-C₆ alkyl, C₃-C₆ cycloalkyl, or cyclized to form a ring where each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, hAr, where hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. The effective dose may have a concentration of between about 20 μM and about 40 μM. The effective dose may have a concentration of between about 40 μM and about 60 μM. The effective dose may have a concentration of between about 60 μM and about 80 μM. The effective dose may have a concentration of between about 80 μM and about 100 μM. The effective dose may have a concentration of between about 100 μM and about 120 μM. The effective dose may have a concentration of between about 100 μM and about 140 μM.

Another general aspect is a method for treatment of a patient. The method includes administering an effective dose of a compound having the formula

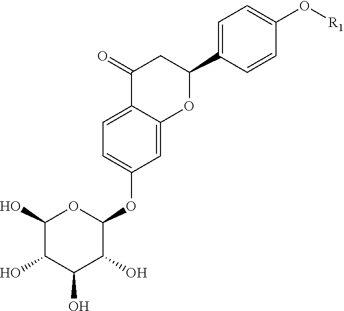

where R₁ includes the group

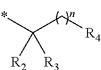

where n is an integer selected from 0 to 5, wherein R₂, R₃, and R₄ are independently selected from the group consisting of: H, C₁-C₆ alkyl, C₃-C₆ cycloalkyl, or cyclized to form a ring where each ring contains or is substituted with 0-3 heteroatoms, Ar, or hAr, where Ar comprises phenyl independently substituted with 0-5 substituents wherein each substituent is independently C₁-C₆ alkyl, C₃-C₆ cycloalkyl, Ar, or hAr, where hAr comprises a 5-7 membered heteroaryl ring independently substituted with 0-6 substituents comprising C₁₋₆ Alk containing 0-3 heteroatoms, Ar, hAr substituted with Alk, OAlk, NHAlk, N(Alk)₂, or a halogen. R₁ may further include the group

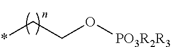

where R₂ and R₃ are independently selected from the group consisting of H, C₁-C₆ alkyl, C₃-C₆ cycloalkyl, Ar, hAr, Na⁺, or K⁺. R₁ may further include the group

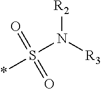

wherein R₂ and R₃ are independently selected from the group consisting of: H, C₁-C₆ alkyl, C₃-C₆ cycloalkyl, or cyclized to form a ring wherein each ring contains or is substituted with 0-3 heteroatoms, Ar, or hAr, where Ar comprises phenyl independently substituted with 0-5 substituents wherein each substituent is independently C₁-C₆ alkyl, C₃-C₆ cycloalkyl, Ar, hAr, wherein hAr comprises a 5-7 membered heteroaryl ring independently substituted with 0-6 substituents comprising C₁₋₆ Alk containing 0-3 heteroatoms, Ar, or hAr substituted with Alk, OAlk, NHAlk, N(Alk)₂, or a halogen. The effective dose may have a concentration of between about 40 μM and about 60 μM. The effective dose may have a concentration of between about 60 μM and about 80 μM. The effective dose may have a concentration of between about 80 μM and about 100 μM. The effective dose may have a concentration of between about 100 μM and about 120 μM. The effective dose may have a concentration of between about 100 μM and about 140 μM.

An exemplary embodiment is a method for treatment of a patient. The method includes administering an effective dose of a compound having the formula

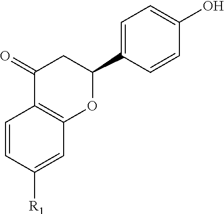

where R₁ is selected from the group consisting of:

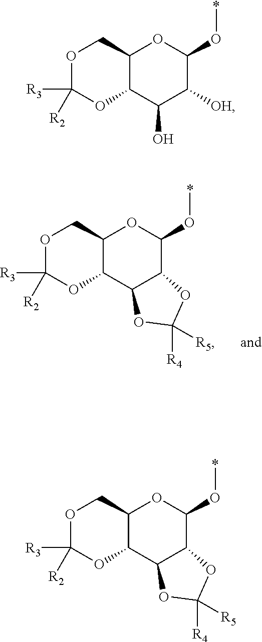

where R₂₋₅ are independently selected from the group consisting of H, C₁₋₆ Alk, C₃₋₆ cycloalk, Ar, or hAr. Alternatively, R₁ may comprise

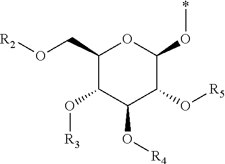

where R₂₋₅ are independently selected from the group consisting of H, C₁₋₆Alk, C₃₋₆ cycloalk, Ar, hAr, or a group selected from: I.

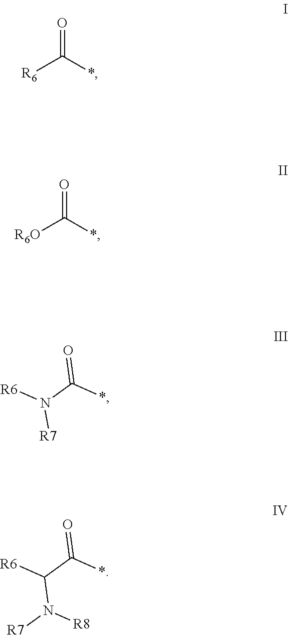

where R₆₋₈ are independently selected from the group consisting of H, C₁₋₆ Alk, C₃₋₆ cycloalk, Ar, or hAr, wherein Ar comprises phenyl independently substituted with 0-5 substituents where each substituent is independently C₁-C₆ alkyl, C₃-C₆ cycloalkyl, Ar, or hAr, where hAr comprises a 5-7 membered heteroaryl ring independently substituted with 0-6 substituents comprising _{C1-6} Alk containing 0-3 heteroatoms, Ar, hAr substituted with Alk, OAlk, NHAlk, N(Alk)₂, or a halogen. The effective dose may have a concentration of between about 40 μM and about 60 μM. The effective dose may have a concentration of between about 60 μM and about 80 μM. The effective dose may have a concentration of between about 80 μM and about 100 μM. The effective dose may have a concentration of between about 100 μM and about 120 μM. The effective dose may have a concentration of between about 100 μM and about 140 μM.

### BRIEF DESCRIPTION OF THE DRAWINGS

FIG. 1 shows various likely and possible target ester prodrug compounds.

FIG. 2 shows various novel target ester prodrug compounds.

FIG. 3 is a bar graph showing a percentage of Aβ fibrillization as compared to a control for sinapic acid O-glucoside samples of various concentrations.

FIG. 4 is a bar graph showing a percentage of survival of SH-SY5Y cells as compared to a control for sinapic acid O-glucoside samples of various concentrations.

FIG. 5 is a bar graph showing a percentage of tau fibrillization as compared to a sample containing Sinapic acid O-glucoside.

FIG. 6 is a bar graph showing a percentage of survival of SH-SY5Y cells in solutions of tau protein and various concentrations of sinapic acid O-glucoside.

FIG. 7 is a bar graph showing a percentage of Aβ fibrillization as compared to a control for neoliquiritin, sebiricose A5, (Z)SA glycoside, and (E)SA glycoside.

FIG. 8 is a bar graph showing a percentage of Aβ fibrillization as compared to a control for neoliquiritin samples of various concentrations.

FIG. 9 is a bar graph showing a percentage of Aβ fibrillization as compared to a control for neoliquiritin and liquiritigenin samples of various concentrations.

FIG. 10 is a bar graph showing a percentage of survival of SH-SY5Y cells in solutions of Aβ and various concentrations of neoliquiritin.

FIG. 11 is a bar graph showing a percentage of survival of SH-SY5Y cells in solutions of neoliquiritin, isoliquiritigenin, sinapic acid O-glucoside, trans-sinapic acid, and liquiritigenin at various concentrations.

FIG. 12 is a bar graph showing a percentage of Aβ fibrillization as compared to a control for sinapic acid, trans-sinapic acid, and sinapic acid O-glucoside at various concentrations.

FIG. 13 is a bar graph showing a percentage of survival of SH-SY5Y cells in solutions of tau protein with neoliquiritin at various concentrations.

FIG. 14 shows a set of bar graphs of measured relative fluorescence units of the effect of Prefibrillated Abeta in the presence of sinapic acid O-glucoside, trans-sinapic acid, and neoliquiritin for various timed treatments.

### DETAILED DESCRIPTION

The disclosed subject matter relates to a pharmaceutical composition containing the natural compound sinapic acid O-glucoside that can be effectively used as a pharmaceutical composition or health functional food to prevent, treat, or improve neurodegeneration or neuroinflammatory diseases. The pharmaceutical composition for preventing or treating neurodegeneration or neuroinflammatory diseases comprises: (A) sinapic acid O-glucoside as an active ingredient (B) ProDrug version of the active ingredient (C) any composition of sinapic acid O-glucoside as an active primary ingredient (D) sinapic acid O-glucoside analog or pharmacol, acceptable salt, solvate, ester, amide, clathrate, stereoisomer, enantiomer, prodrug or analog thereof, or a combination thereof.

Several studies have suggested that reducing Aβ aggregation, or promoting its clearance from the brain, could potentially improve neuronal survival and alleviate some symptoms of Alzheimer's disease. For instance, many experimental Alzheimer's disease treatments aim to reduce Aβ levels in the brain, either by preventing Aβ production, inhibiting its aggregation, or promoting its clearance.

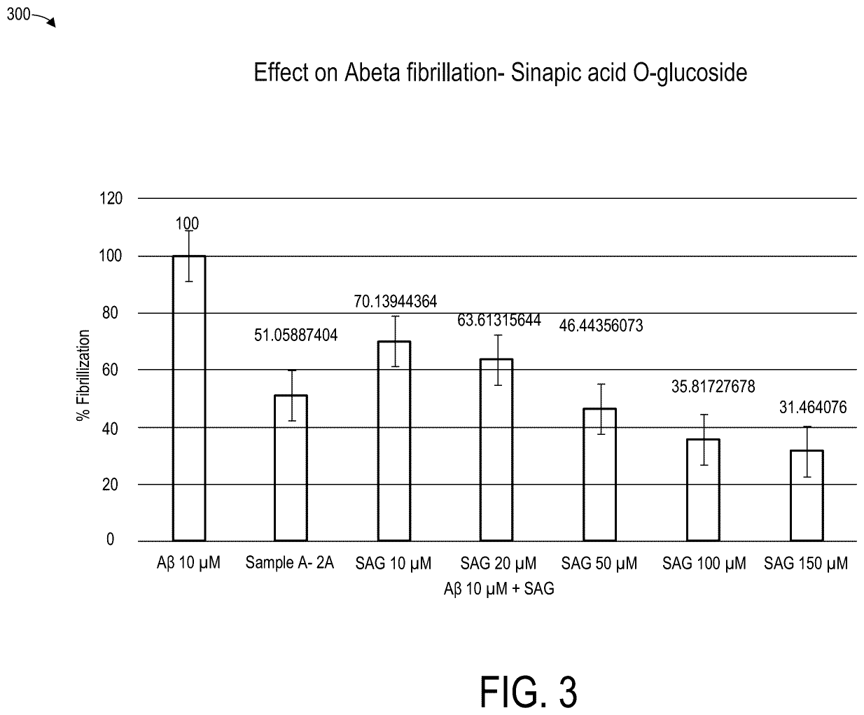

However, it's important to note that while reducing Aβ fibril formation could theoretically help improve neuronal survival, this is only one aspect of a complex disease process. Other factors, such as tau protein aggregation, inflammation, oxidative stress, and vascular factors, also play a role in the development and progression of Alzheimer's disease. A disclosed treatment includes treating a patient with an effective dose of the disclosed compositions. The term effective dose, as used herein, may include a dose capable of improving a factor indicative of neural survival, neural inflammation, cellular toxicity, Aβ fibril formation, tau protein aggregation, inflammation, oxidative stress, and vascular factors. Examples of factors that may be improved by an effective dose from one or more of the disclosed compositions are disclosed in results/data in FIGS. 3-14.

### (E)-3-[3,5-dimethoxy-4-[(2S,3R,4S,5S,6R)-3,4,5-trihydroxy-6-(hydroxymethyl)oxan-2-yl]oxyphenyl]prop-2-enoic acid (sinapic acid O-glucoside=SAG) has the structure

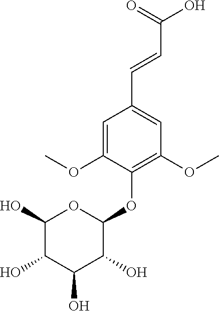 [SMILES: COc1cc(/C=C/C(=O)O)cc(OC)c1O[C@@H]1O[C@H](O)[C@@H](O)[C@H](O)[C@H]1O]

and has been shown to reduce Aβ fibrillization. Another compound, Neoliquiritin, which has the structure

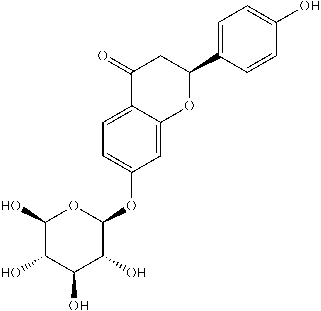 [SMILES: O=C1C[C@@H](c2ccc(O)cc2)Oc2cc(O[C@@H]3O[C@H](O)[C@@H](O)[C@H](O)[C@H]3O)ccc21]

and has been shown to reduce Aβ fibrillization. SAG and Neoliquiritin, which are referred to as the parent compounds, have shown promise as therapeutic agents, but their clinical application is limited by its suboptimal bioavailability and restricted brain penetration. The polar functional groups present in the parent compound hinder its ability to passively diffuse through physiological membranes, thus impeding its distribution and efficacy.

The disclosed subject matter includes novel prodrugs with reduced polarity that offer significant improvements compared to the parent compounds, including: (1) Improved passive permeability. This feature facilitates their efficient diffusion through various physiological membranes, including the intestines, target organ cell membranes, and the blood-brain barrier. (2) Increased bioavailability. Higher passive permeability allows the prodrugs achieve greater bioavailability, allowing for higher concentrations to be delivered to the intended site(s) of action. (3) Improved brain penetration. In a similar fashion, increased passive permeability will allow for greater passage through the blood-brain barrier, potentially enabling enhanced drug delivery to the central nervous system for therapeutic applications. (4) Select examples of the disclosed prodrugs are likely substrates for endogenous transporters expressed in diverse cell types throughout the body. This characteristic augments their permeability across various physiological barriers, expanding their therapeutic potential.

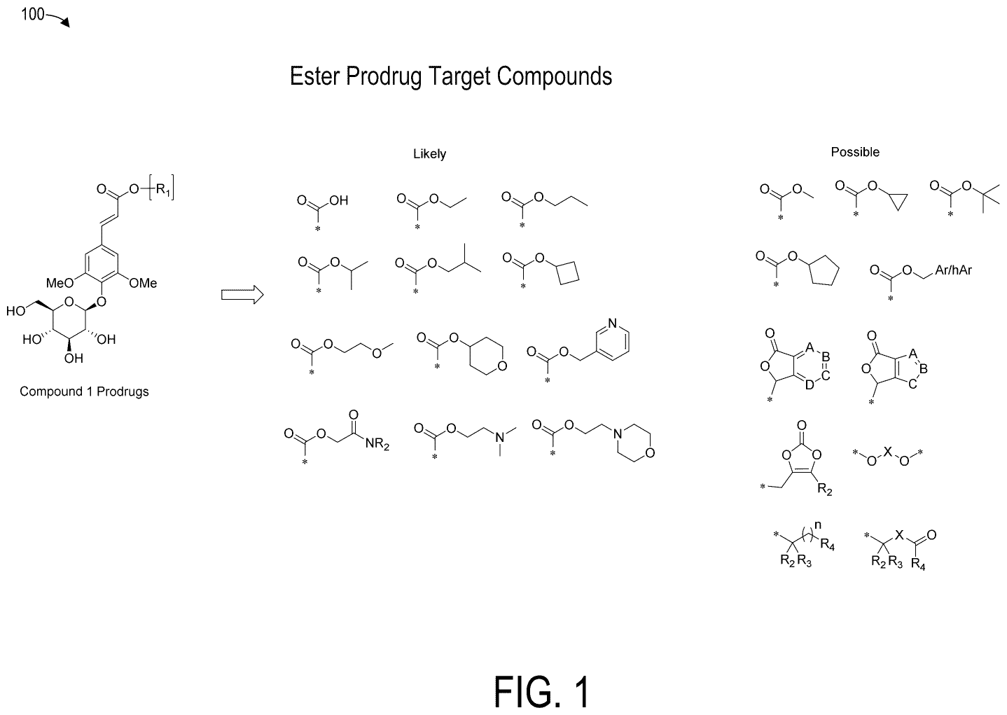

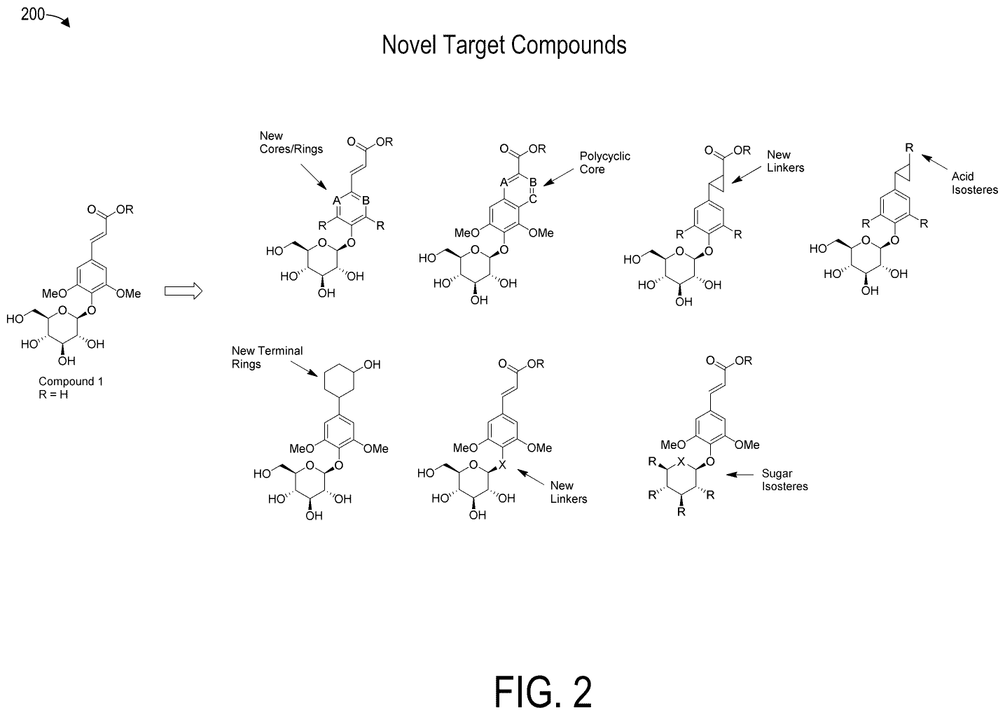

Referring to FIG. 1 and FIG. 2, FIG. 1 is a chart 100 showing examples of likely and possible prodrugs for and ester of SAG. FIG. 2 is a chart 200 that shows novel target compounds of prodrug that deliver SAG.

Examples of prodrugs for SAG may include the following structure

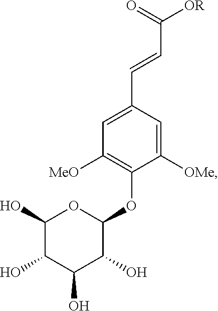

herein referred to as parent compound (1).

In various embodiments, R may include, but is not limited to any of the following groups:

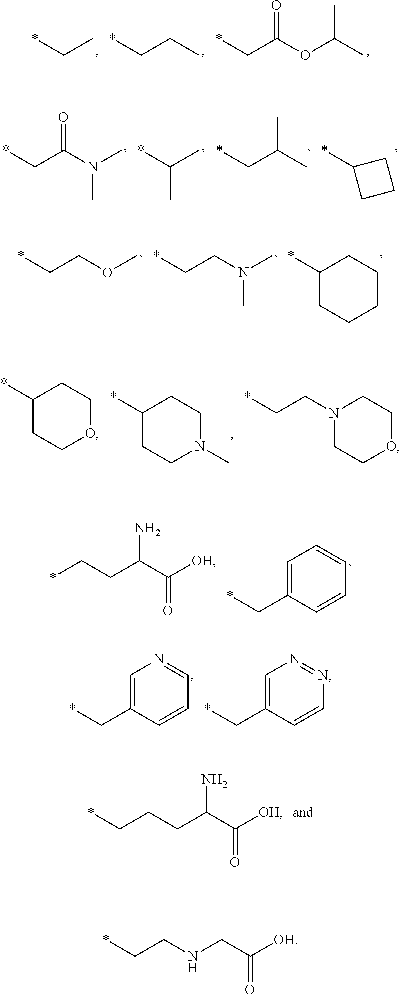

Another example of a prodrug for SAG may include a sugar prodrug the following structure:

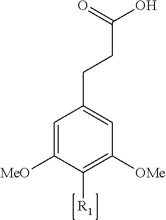

In various embodiments, R₁ may include, but is not limited to any of the following groups:

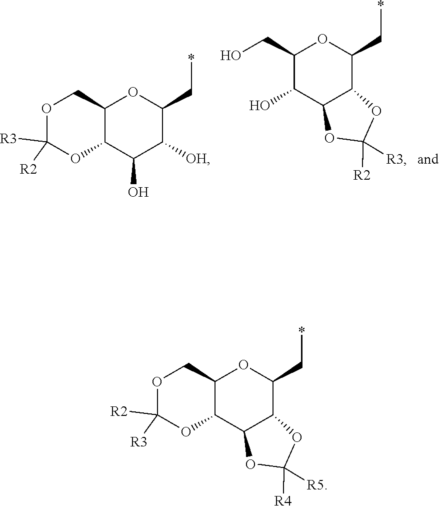

R₂₋₅ may be independently selected from the following: H, C₁₋₆Alk, C₃₋₆ cycloalk, Ar, or hAr. Alternatively, R₁ may include, but is not limited to any of the following groups:

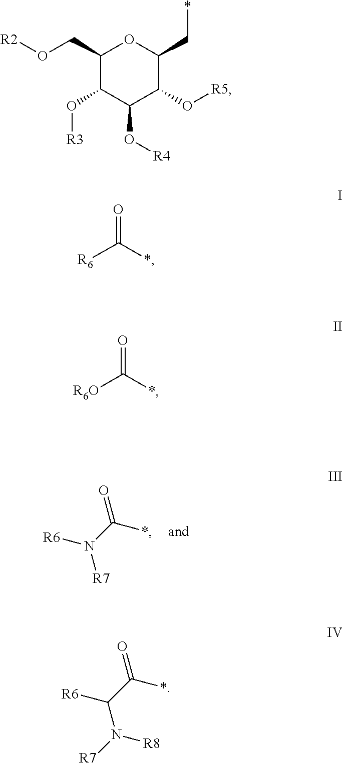

R₆₋₈ may be independently selected from the following: H, C₁₋₆ Alk, C₃₋₆ cycloalk, Ar, or hAr. Ar may include phenyl independently substituted with 0-5 substituents where each substituent is independently C₁-C₆ alkyl, C₃-C₆ cycloalkyl, Ar, hAr(alkyl, O-alkyl, NH-alkyl, or N(alkyl)₂. hAr may include a 5-7 membered heteroaryl ring independently substituted with 0-6 substituents comprising _{C1-6} Alk containing 0-3 heteroatoms, Ar, hAr substituted with (Alk, OAlk, NHAlk, N(Alk)₂, or a halogen.

An example of a prodrug for neoliquiritin may comprise that following structure:

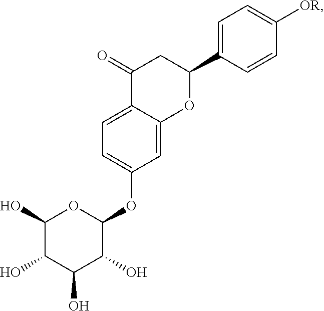

herein referred to as parent compound (2).

In various embodiments, a prodrug with the following structure

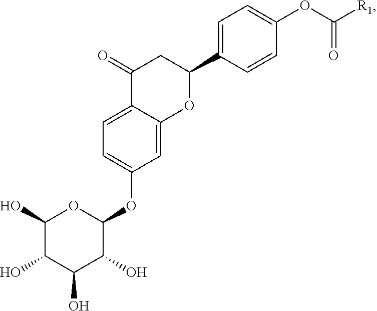

herein referred to as parent compound (3), may be administered to a patient for treatment of a cognitive disorder such as Alzheimer's disease.

In various embodiments, a prodrug with the following structure

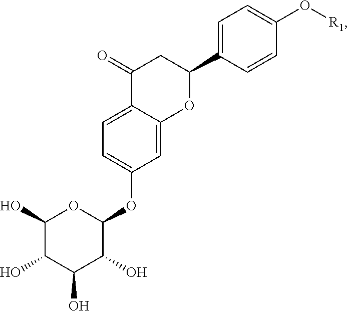

herein referred to as parent compound (4), may be administered to a patient for treatment of a cognitive disorder such as Alzheimer's disease.

In various embodiments, a first synthetic route for parent compound (1) comprises:

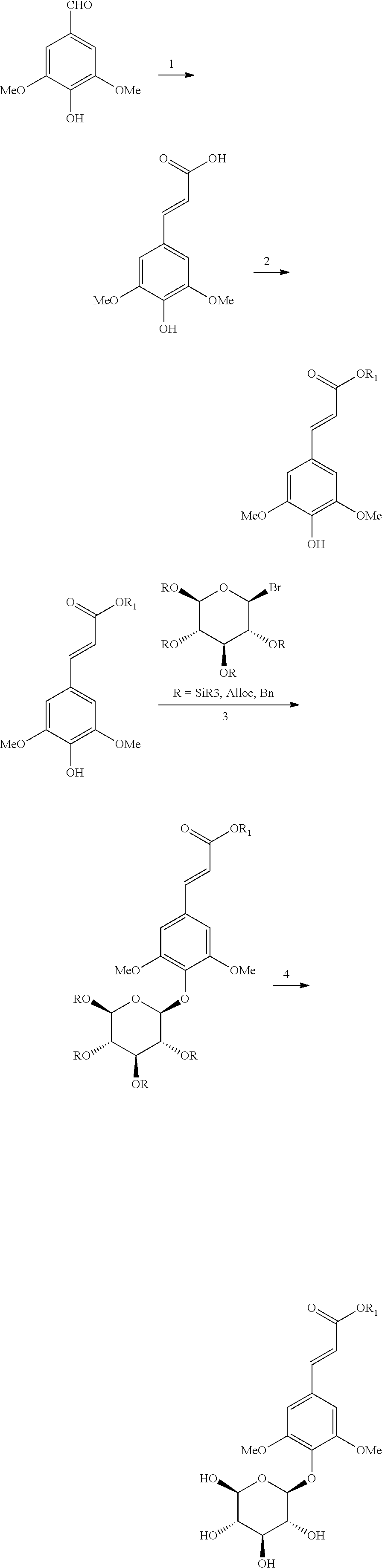

where step 1 comprises reacting with malonic acid, piperzine, and toluene at 80 C; step 2 comprises reacting with sulfuric acid and alcohol at 75 C, step 3 comprises reacting with TBAB and 20% K₂CO₃ at 65 C; and step 4 comprises reacting with TBAF or Pd(Ph)₄ or H₂.

A second synthetic route for parent compound (1) comprises:

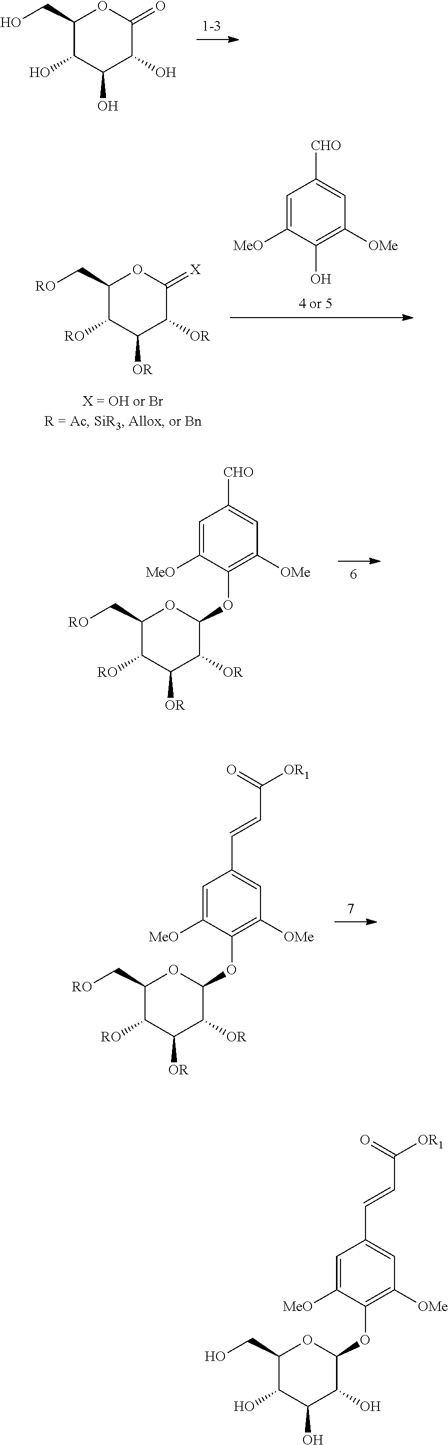

where step 1 comprises reacting with AcCl or SiR_{3Cl} or AllocCl or BnCl in basic conditions; step 2 comprises reacting with H₂ and Pd/C (X═OH); step 3 comprises reacting with POBr₃ (X═Br); step 4 comprises reacting with TBAB and K₂CO₃ (X═OH); step 5 comprises reacting with K₂CO₃ and DMA (X═Br); step 6 comprises reacting with piperazine and toluene at 80 C; and step 7 comprises reacting with NaOH or TBAF or PdPh₄ or H₂ and Pd/C.

A third synthetic route for parent compound (1) comprises:

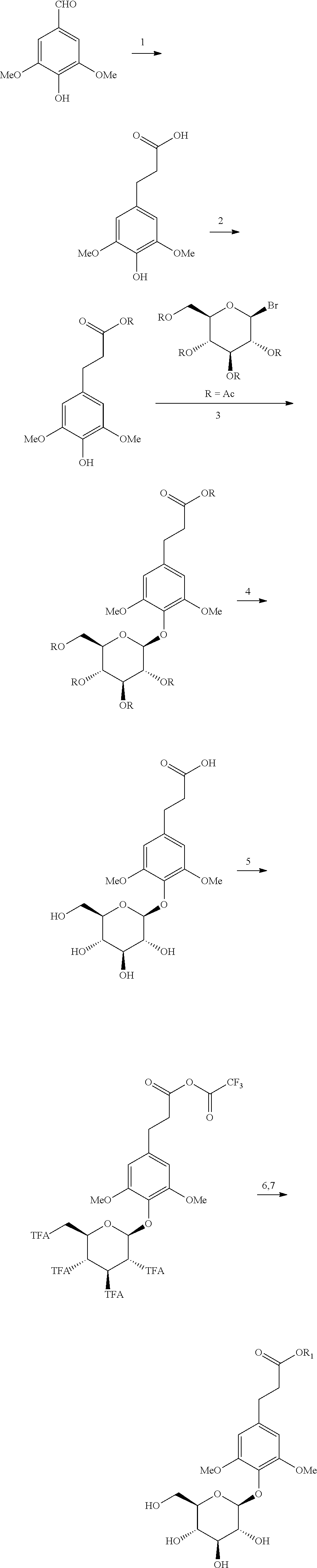

where step 1 comprises reacting with malonic acid, piperazine, p-aminotoluene, and toluene at 80 C; step 2 comprises reacting with sulfuric acid and alcohol at 75 C; step 3 comprises reacting with TBAB and 20% K₂CO₃ at 65 C; step 4 comprises reacting with NaOH, H₂₀, and EtOH; step 5 comprises reacting with TFAA in basic conditions; step 6 comprises reacting with R₁OH in basic conditions; and step 7 comprises reacting with 0.1% NaOH, H₂O, and DMA.

Referring to FIG. 3, FIG. 3 is a bar graph 300 showing a percentage of Aβ fibrillization as compared to a control for various samples. The left side of the bar graph shows a sample of Aβ protein at μM. Fibrillization was normalized to the left sample at 100%. All other samples were added to the solution with 10 μM Aβ and fibrillization of the sample was measured.

Sample A, which is a substance based on a traditional treatment, showed significant decrease in fibrillization at 51%. Sample A was subsequently fractionated to identify one or more compounds in Sample A. Accordingly, Sample A was fractionated into polar and nonpolar fractions. 4540 compounds were identified in the nonpolar fraction using a C18 column in the Ometa platform. The bioactivity was around 30%. 2825 compounds were identified in the polar fraction using a HILIC column in the Ometa platform. The bioactivity was around 50%. The compounds found to be responsible in the polar fraction are sinapic acid 0-glycoside and neoliquiritin.

Accordingly, the rest of the graph shown in FIG. 3 shows a percent fibrillization for sinapic acid O-glycoside (SAG) at various concentrations in the solution of 10 μM Aβ. The solution of 10 μM Aβ with 10 μM SAG was shown to have a fibrillation of 70% as compared to the control. The solution of 10 μM Aβ with 20 μM SAG was shown to have a fibrillation of 64% as compared to the control. The solution of 10 μM Aβ with 50 μM SAG was shown to have a fibrillation of 46% as compared to the control. The solution of 10 μM Aβ with 100 μM SAG was shown to have a fibrillation of 36% as compared to the control. The solution of 10 μM Aβ with 150 μM SAG was shown to have a fibrillation of 31% as compared to the control.

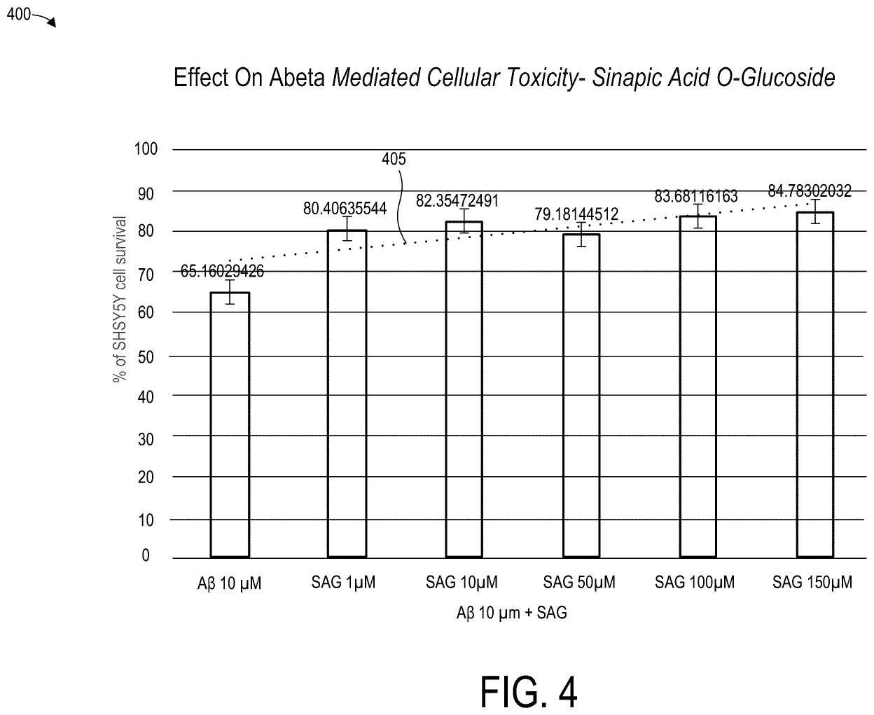

Referring to FIG. 4, FIG. 4 is a bar graph 400 showing a percentage of survival of SH-SY5Y cells as compared to a control for sinapic acid O-glucoside samples of various concentrations. The left side of the graph shows a percentage of survival for a control solution of 10 μM Aβ. As shown in the bar graph 400, the control solution has a percent survival of 65%. All other samples shown on the bar graph 400 comprise a solution of 10 μM Aβ and SAG at a variable concentration.

The solution of 10 μM Aβ and 1 μM SAG showed a cell survival of 80%. The solution of 10 μM Aβ and 10 μM SAG showed a cell survival of 82%. The solution of 10 μM Aβ and 50 μM SAG showed a cell survival of 79%. The solution of 10 μM Aβ and 100 μM SAG showed a cell survival of 84%. And the solution of 10 μM Aβ and 150 μM SAG showed a cell survival of 85%. A trendline 405 on the bar graph 400 indicates that increasing a concentration of SAG is positively correlated with an increase in cell survival.

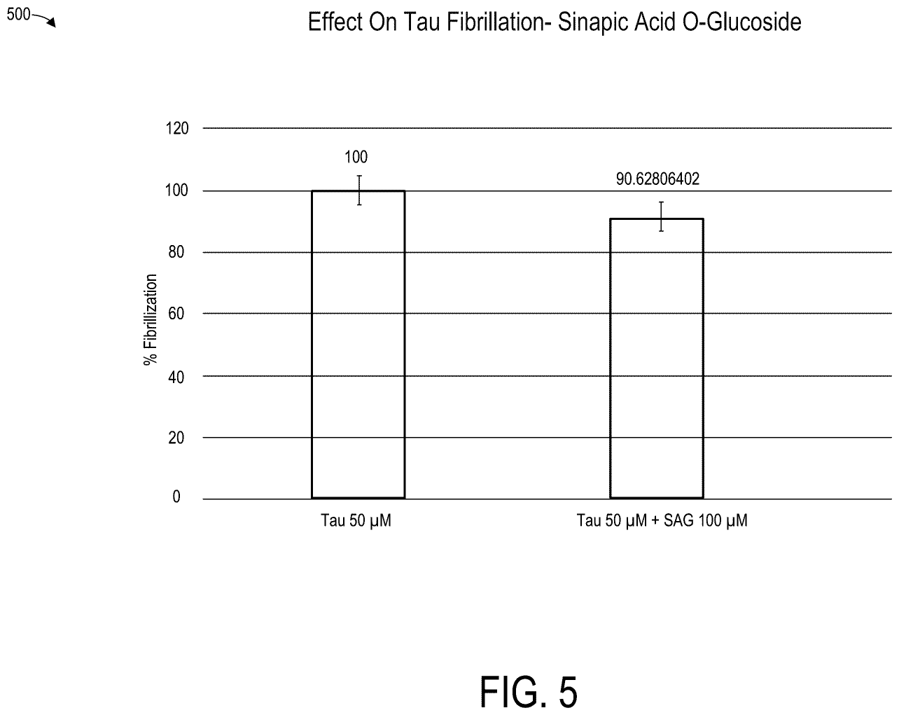

Referring to FIG. 5, FIG. 5 is a bar graph 500 showing a percentage of tau fibrillization as compared to a sample containing sinapic acid O-glucoside. The bar graph 500 shows a percent of tau protein fibrillization. A control sample, containing 50 μmol of tau protein, has a percent fibrillization normalized to 100. A sample containing 50 μmol tau protein and 50 μmol SAG was shown to have 91% tau protein fibrillization as compared to the control containing just 50 μmol tau protein.

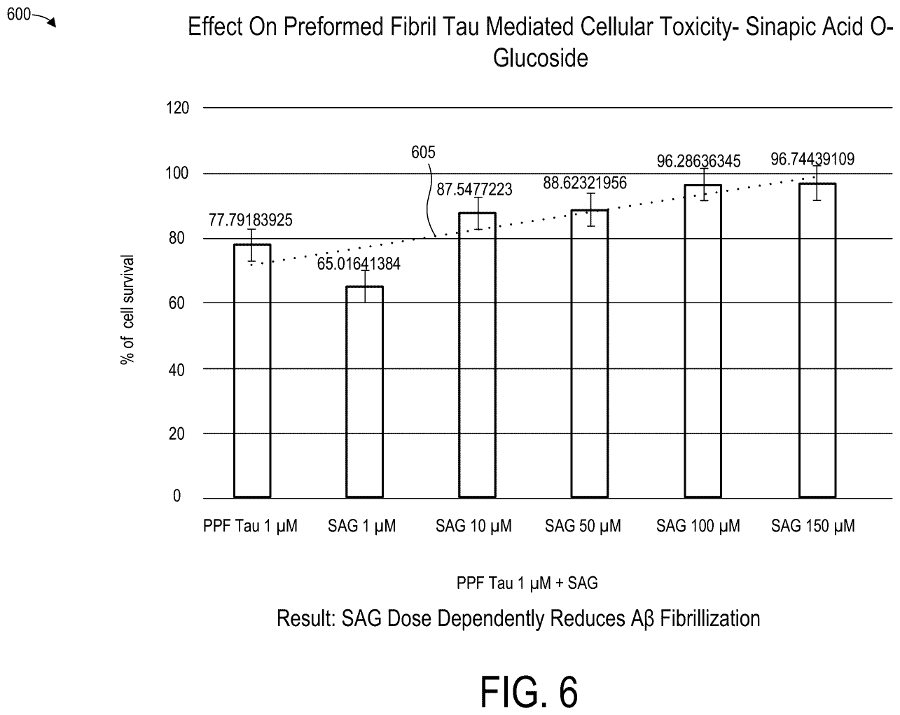

Referring to FIG. 6, FIG. 6 is a bar graph 600 showing a percentage of survival of SH-SY5Y cells in solutions of tau protein and various concentrations of sinapic acid O-glucoside. A control sample comprising a sample of SH-SY5Y cells with preformed fibrils (PPF) of tau protein was prepared. The percentage of cell survival was measured at 78% for the control sample. The rest of the samples on the bar graph 600 include a variable concentration of SAG as well as the SH-SY5Y cells and 1 μM of PPF tau protein.

The sample comprising 1 μM SAG, 1 μM of PPF tau protein, and SH-SY5Y cells had a cell survival of 65%. The sample comprising 10 μM SAG, 1 μM of PPF tau protein, and SH-SY5Y cells had a cell survival of 88%. The sample comprising 50 μM SAG, 1 μM of PPF tau protein, and SH-SY5Y cells had a cell survival of 89%. The sample comprising 100 μM SAG, 1 μM of PPF tau protein, and SH-SY5Y cells had a cell survival of 96%. The sample comprising 150 μM SAG, 1 μM of PPF tau protein, and SH-SY5Y cells had a cell survival of 97%. A trendline 605 in the bar graph 600 indicates a positive correlation between concentration of SAG and survival of SH-SY5Y cells.

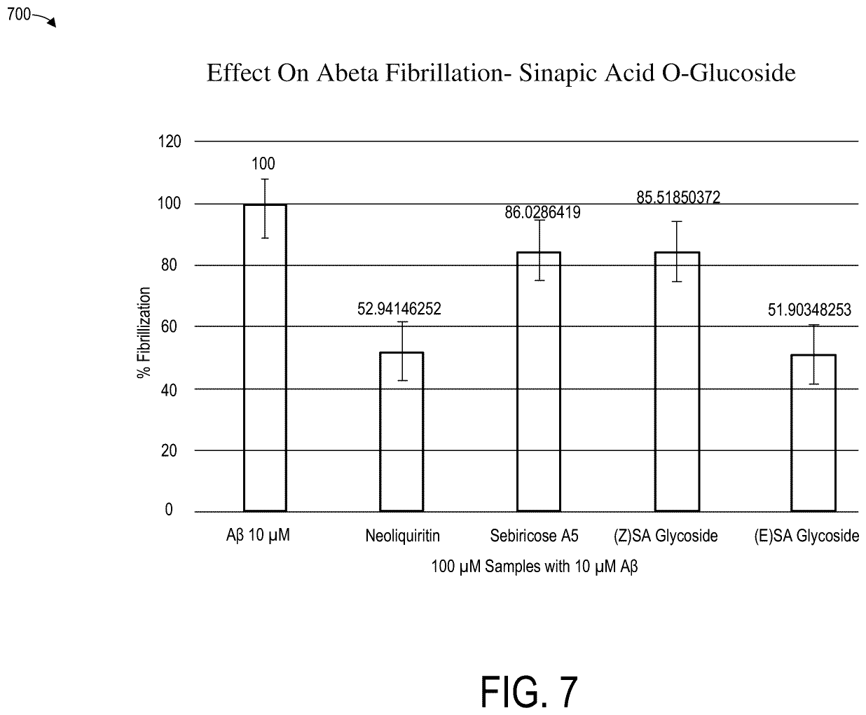

Referring to FIG. 7, FIG. 7 is a bar graph 700 showing a percentage of Aβ fibrillization as compared to a control for neoliquiritin, sebiricose A5, (Z)SA glycoside, and (E)SA glycoside. The bar graph 700 includes a control sample with 10 μM Aβ protein. The amount of Aβ fibrillization in the control sample is normalized to 100%. All other samples in the bar graph 700 include 10 μM Aβ protein and another compound at a concentration of 100 μM.

The sample with 10 μM Aβ protein and 100 μM neoliquiritin was shown to have a 53% fibrillization as compared to the control. The sample with 10 μM Aβ protein and 100 μM Sebiricose A5 was shown to have 86% fibrillization as compared to the control. The sample with 10 μM Aβ protein and 100 μM (Z)SA glycoside was shown to have 86% fibrillization as compared to the control. The sample with 10 μM Aβ protein and 100 μM (E)SA glycoside was shown to have a 51% fibrillization as compared to the control. Accordingly neoliquiritin and (E)SA glycoside were shown to effectively reduce Aβ fibrils when compared to Sebiricose A5 and (Z)SA glycoside.

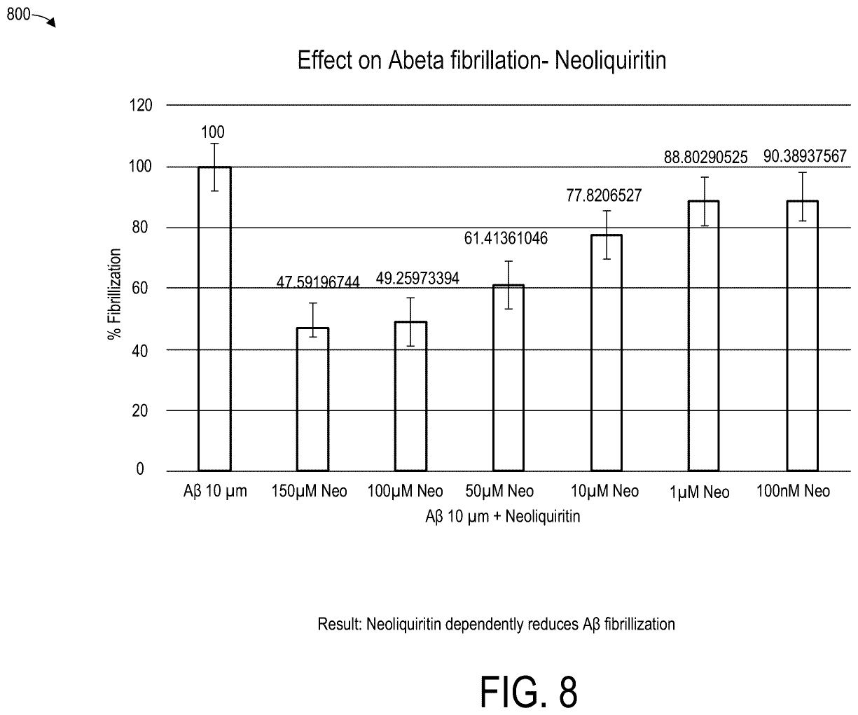

Referring to FIG. 8, FIG. 8 is a bar graph 800 showing a percentage of Aβ fibrillization as compared to a control for neoliquiritin samples of various concentrations. The amount of Aβ fibrillization in the control sample is normalized to 100%. All other samples in the bar graph 800 include 10 μM Aβ protein and a variable concentration of neoliquiritin.

As shown in the bar graph 800, the sample with 10 μM Aβ protein and 150 μM neoliquiritin was shown to have a 48% fibrillization of Aβ protein as compared to the control. The sample with 10 μM Aβ protein and 100 μM neoliquiritin was shown to have a 49% fibrillization of Aβ protein as compared to the control. The sample with 10 μM Aβ protein and 50 μM neoliquiritin was shown to have a 61% fibrillization of Aβ protein as compared to the control. The sample with 10 μM Aβ protein and 10 μM neoliquiritin was shown to have a 78% fibrillization of Aβ protein as compared to the control. The sample with 10 μM Aβ protein and 1 μM neoliquiritin was shown to have a 89% fibrillization of Aβ protein as compared to the control. The sample with 10 μM Aβ protein and 100 nM neoliquiritin was shown to have a 90% fibrillization of Aβ protein as compared to the control. Accordingly, it was found that neoliquiritin reduces Aβ fibrillization.

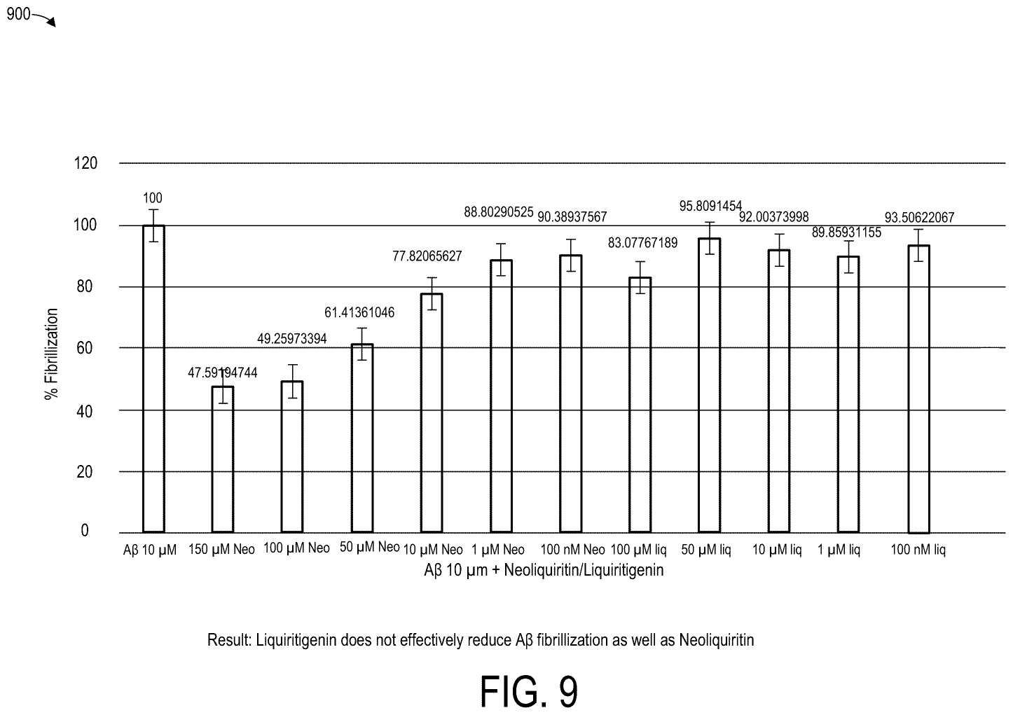

Referring to FIG. 9, FIG. 9 is a bar graph 900 showing a percentage of Aβ fibrillization as compared to a control for neoliquiritin and liquiritigenin samples of various concentrations. Like the other bar graphs, the control sample is on the left side of the bar graph 900. The control sample comprises 10 μM Aβ protein and the percent fibrillization of Aβ protein in the control sample is normalized to 100. All other samples, which contain either neoliquiritin or liquiritigenin, also include a concentration of 10 μM Aβ protein.

The sample containing 150 μM neoliquiritin and 10 μM Aβ protein was found to have 48% fibrillization of Aβ protein. The sample containing 100 μM neoliquiritin and 10 μM Aβ protein was found to have 49% fibrillization of Aβ protein. The sample containing 50 μM neoliquiritin and 10 μM Aβ protein was found to have 61% fibrillization of Aβ protein. The sample containing 10 μM neoliquiritin and 10 μM Aβ protein was found to have 77% fibrillization of Aβ protein. The sample containing 1 μM neoliquiritin and 10 μM Aβ protein was found to have 89% fibrillization of Aβ protein. The sample containing 100 nM neoliquiritin and 10 μM Aβ protein was found to have 89% fibrillization of Aβ protein.

The sample containing 100 μM liquiritigenin and 10 μM Aβ protein was found to have 83% fibrillization of Aβ protein. The sample containing 50 μM liquiritigenin and 10 μM Aβ protein was found to have 97% fibrillization of Aβ protein. The sample containing 10 μM liquiritigenin and 10 μM Aβ protein was found to have 92% fibrillization of Aβ protein. The sample containing 10 μM liquiritigenin and 10 μM Aβ protein was found to have 90% fibrillization of Aβ protein. The sample containing 100 nM liquiritigenin and 10 μM Aβ protein was found to have 94% fibrillization of Aβ protein. Accordingly, the bar graph 900 shows that liquiritigenin does not effectively reduce Aβ fibrillization as well as neoliquiritin.

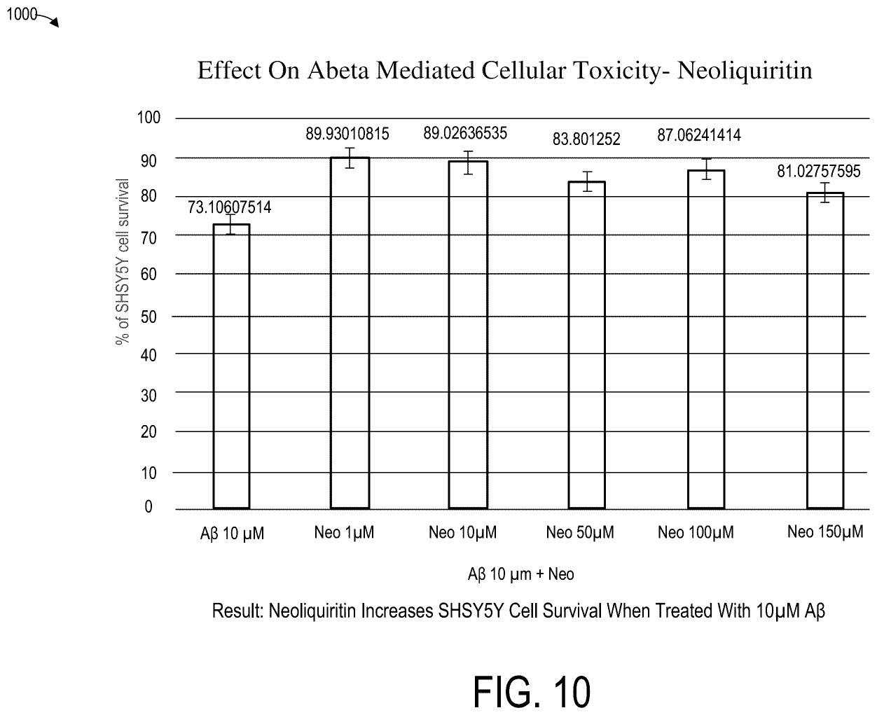

Referring to FIG. 10, FIG. 10 is a bar graph 1000 showing a percentage of survival of SH-SY5Y cells in solutions of Aβ and various concentrations of neoliquiritin. Cell survival of SH-SY5Y cells for the control sample, comprising 10 μM Aβ protein, was shown to be 73%. All the samples in the bar graph 1000 comprised the same concentration of 10 μM Aβ protein as the control sample as well as a variable concentration of neoliquiritin.

Starting from the left side of the bar graph 1000, the sample containing 10 μM Aβ protein and 1 μM neoliquiritin had a 90% survival rate for the SH-SY5Y cells. The sample containing 10 μM Aβ protein and 10 μM neoliquiritin had an 89% survival rate for the SH-SY5Y cells. The sample containing 10 μM Aβ protein and 50 μM neoliquiritin had an 84% survival rate for the SH-SY5Y cells. The sample containing 10 μM Aβ protein and 100 μM neoliquiritin had an 87% survival rate for the SH-SY5Y cells. The sample containing 10 μM Aβ protein and 150 μM neoliquiritin had an 81% survival rate for the SH-SY5Y cells.

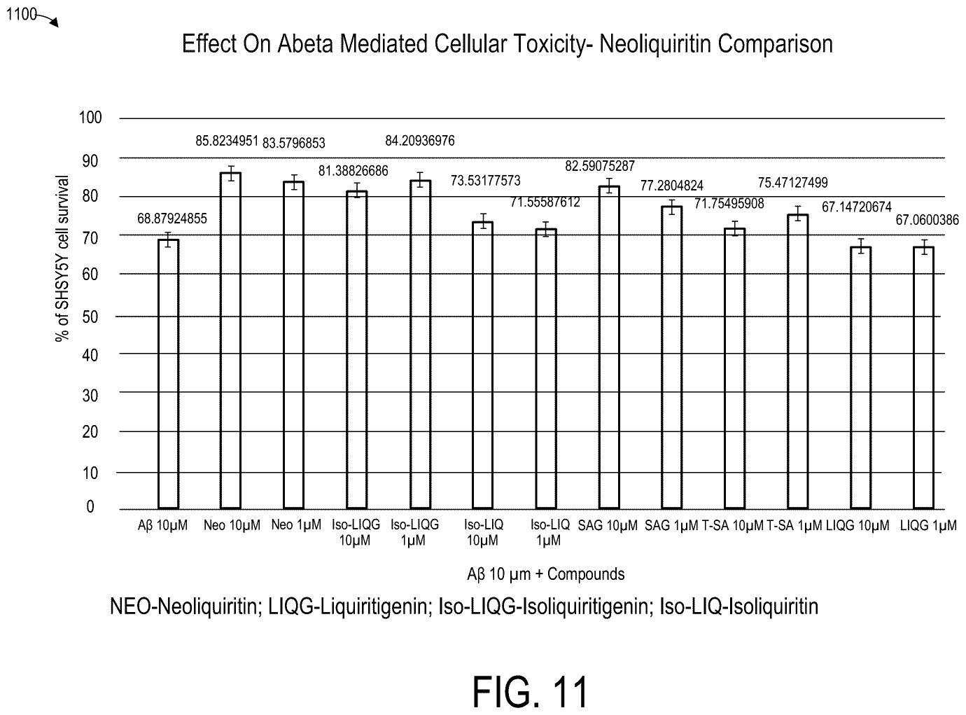

Referring to FIG. 11, FIG. 11 is a bar graph 1100 showing a percentage of survival of SH-SY5Y cells in solutions of neoliquiritin, isoliquiritigenin, isoliquiritin, sinapic acid O-glucoside, trans-sinapic acid, and liquiritigenin at various concentrations. The control sample, containing 10 μM Aβ protein, was found to have a 69% survival rate for the SH-SY5Y cells. The other samples in the bar graph 1100 all contain the same concentration of 10 μM Aβ protein as a control sample.

Starting from the left most sample next to the control sample of the bar graph 1100, the sample comprising 10 μM Aβ protein and 10 μM neoliquiritin was shown to have an 86% survival for the SH-SY5Y cells. The sample comprising 10 μM Aβ protein and 1 μM neoliquiritin was shown to have an 84% survival for the SH-SY5Y cells. The sample comprising 10 μM Aβ protein and 10 μM isoliquiritigenin was shown to have an 81% survival for the SH-SY5Y cells. The sample comprising 10 μM Aβ protein and 1 μM isoliquiritigenin was shown to have an 84% survival for the SH-SY5Y cells.

The sample comprising 10 μM Aβ protein and 10 μM isoliquiritin was shown to have a 74% survival for the SH-SY5Y cells. The sample comprising 10 μM Aβ protein and 1 μM isoliquiritin was shown to have a 72% survival for the SH-SY5Y cells. The sample comprising 10 μM Aβ protein and 10 μM SAG was shown to have an 83% survival for the SH-SY5Y cells. The sample comprising 10 μM Aβ protein and 1 μM SAG was shown to have a 77% survival for the SH-SY5Y cells. The sample comprising 10 μM Aβ protein and 1 μM SAG was shown to have a 77% survival for the SH-SY5Y cells.

The sample comprising 10 μM Aβ protein and 10 μM trans-sinapic acid (T-SA) was shown to have a 77% survival for the SH-SY5Y cells. The sample comprising 10 μM Aβ protein and 1 μM T-SA was shown to have a 76% survival for the SH-SY5Y cells. The sample comprising 10 μM Aβ protein and 10 μM liquiritigenin was shown to have a 67% survival for the SH-SY5Y cells. The sample comprising 10 μM Aβ protein and 1 μM liquiritigenin was shown to have a 67% survival for the SH-SY5Y cells.

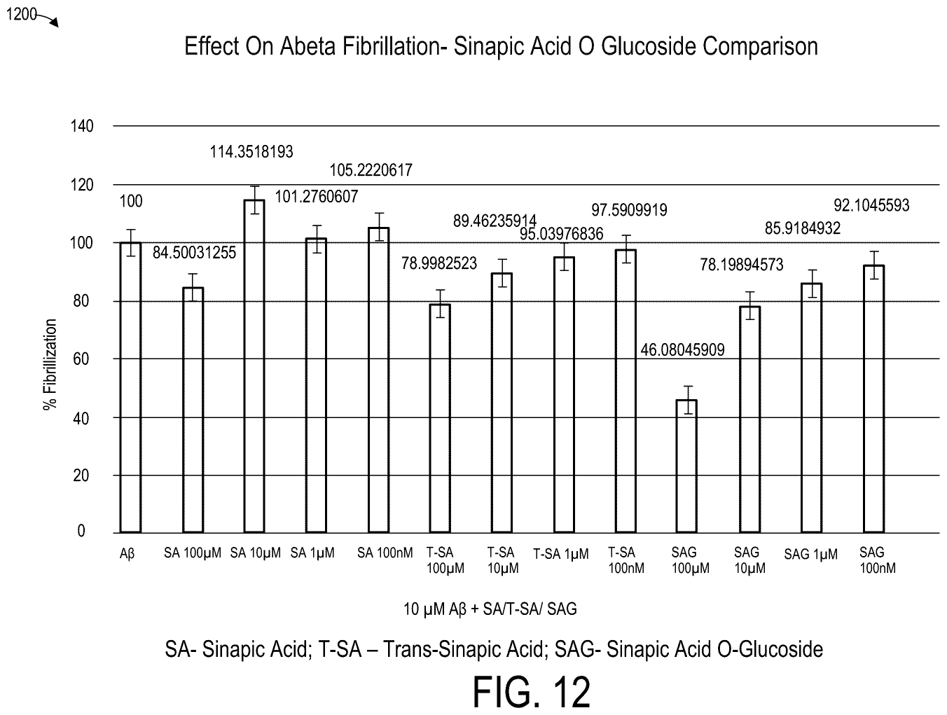

Referring to FIG. 12, FIG. 12 is a bar graph 1200 showing a percentage of Aβ fibrillization as compared to a control for sinapic acid, trans-sinapic acid, and sinapic acid O-glucoside at various concentrations. On the left side of the bar graph 1200, a control sample comprising 10 μM Aβ protein has a fibrillization measurement normalized to 100. All other samples include the same concentration of 10 μM Aβ protein as a control sample.

The sample containing 10 μM Aβ protein and 100 μM sinapic acid (SA) was shown to have an 85% Aβ fibrillization as compared to the control. The sample containing 10 μM Aβ protein and 10 μM SA was shown to have a 114% Aβ fibrillization as compared to the control. The sample containing 10 μM Aβ protein and 1 μM SA was shown to have a 101% Aβ fibrillization as compared to the control. The sample containing 10 μM Aβ protein and 100 nM SA was shown to have a 105% Aβ fibrillization as compared to the control.

The sample containing 10 μM Aβ protein and 100 μM T-SA was shown to have a 79% Aβ fibrillization as compared to the control. The sample containing 10 μM Aβ protein and 10 μM T-SA was shown to have an 89% Aβ fibrillization as compared to the control. The sample containing 10 μM Aβ protein and 1 μM T-SA was shown to have a 95% Aβ fibrillization as compared to the control. The sample containing 10 μM Aβ protein and 100 nM T-SA was shown to have a 98% Aβ fibrillization as compared to the control.

The sample containing 10 μM Aβ protein and 100 μM SAG was shown to have a 46% Aβ fibrillization as compared to the control. The sample containing 10 μM Aβ protein and 10 μM SAG was shown to have a 78% Aβ fibrillization as compared to the control. The sample containing 10 μM Aβ protein and 1 μM SAG was shown to have an 85% Aβ fibrillization as compared to the control. The sample containing 10 μM Aβ protein and 100 nM SAG was shown to have a 92% Aβ fibrillization as compared to the control.

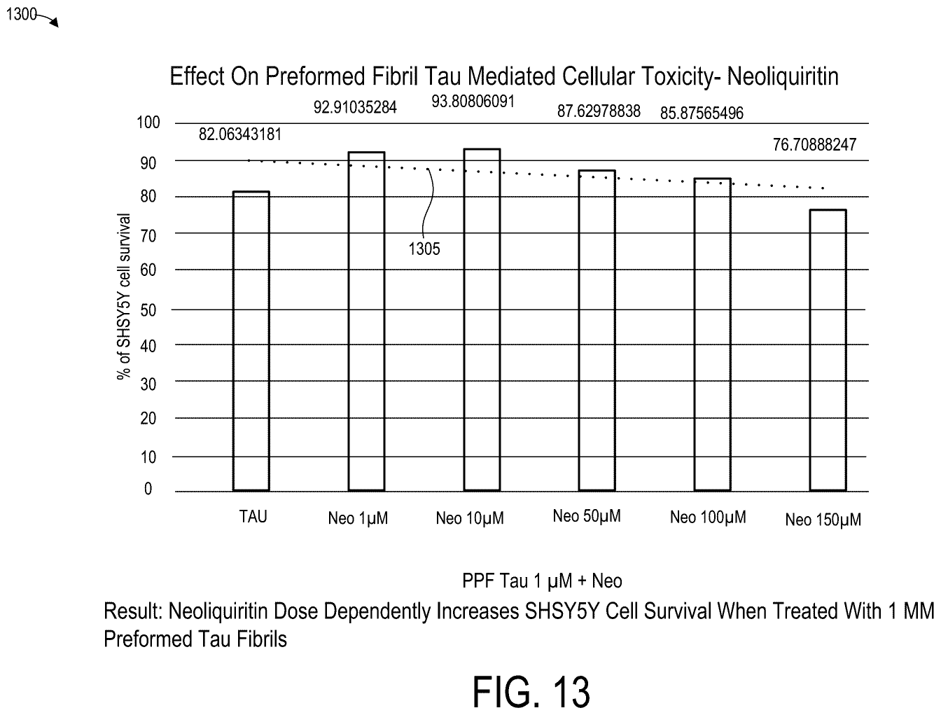

Referring to FIG. 13, FIG. 13 is a bar graph 1300 showing a percentage of survival of SH-SY5Y cells in solutions of tau protein with neoliquiritin at various concentrations. The control sample shown on the left side of the bar graph 1300 comprises a concentration of 1 μM PPF tau protein. All other samples in the bar graph 1300 include the same concentration of preformed tau fibrils as a control sample.

Starting from the left side of the bar graph 1300, the sample containing 1 μM neoliquiritin and 1 μM PPF tau protein was shown to have 93% survival for SH-SY5Y cells. Moving right, the sample containing 10 μM neoliquiritin and 1 μM PPF tau protein was shown to have 94% survival for SH-SY5Y cells. The sample containing 50 μM neoliquiritin and 1 μM PPF tau protein was shown to have 88% survival for SH-SY5Y cells. The sample containing 100 μM neoliquiritin and 1 μM PPF tau protein was shown to have 86% survival for SH-SY5Y cells. The sample containing 150 μM neoliquiritin and 1 μM PPF tau protein was shown to have 78% survival for SH-SY5Y cells. The trend line 1305 in the bar graph 1300 shows that lower concentrations of neoliquiritin are more effective at increasing cell survival.

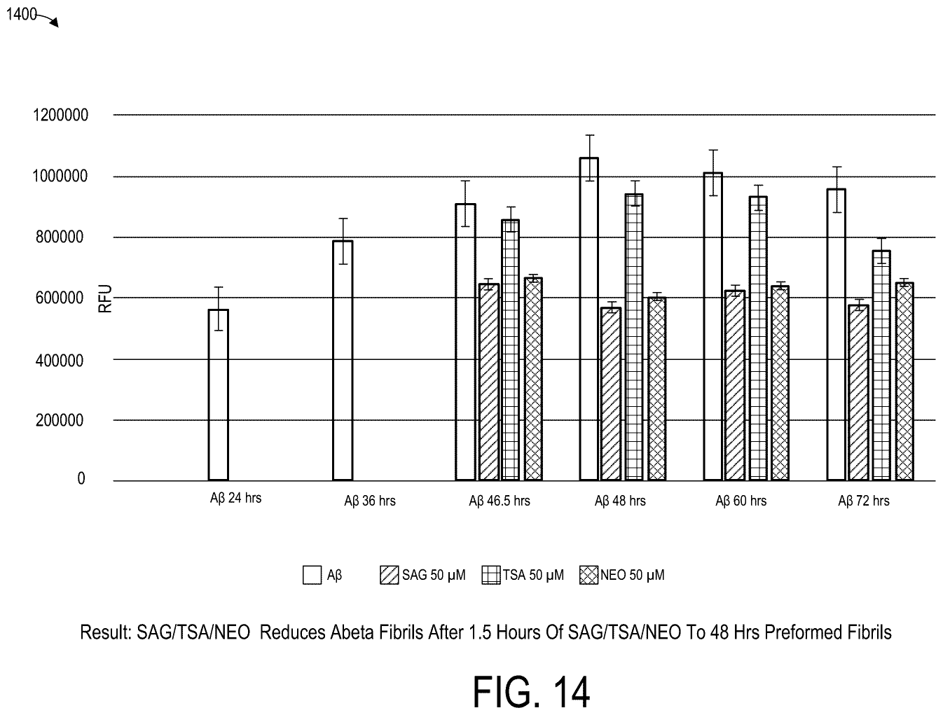

Referring to FIG. 14, FIG. 14 shows a set of bar graphs 1400 of measured relative fluorescence units (RFU) after treatment with solutions of sinapic acid O-glucoside, trans-sinapic acid, and neoliquiritin following various timed treatment with Aβ fibrils. Control samples containing only Aβ protein as starting material are unshaded. Other samples containing Aβ protein and an additional compound have shading according to the legend on the bottom of the bar graphs 1400.

Starting from the left side of the bar graphs 1400, relative fluorescence for Aβ fibrils at 24 hours is approximately 560000 and relative fluorescence for Aβ fibrils at 36 hours is approximately 790000. Relative fluorescence for Aβ fibrils at 46.5 hours is approximately 900000. Relative fluorescence for Aβ fibrils at 46.5 hours treatment with 50 μM SAG was shown to be approximately 640000. Relative fluorescence for Aβ fibrils at 46.5 hours treatment with 50 μM T-SA was shown to be approximately 850000. Relative fluorescence for Aβ fibrils at 46.5 hours with 50 μM neoliquiritin was shown to be approximately 660000.

Moving on to the samples that were subjected to 48 hours of treatment with Aβ protein, the sample of only Aβ protein as a starting material had a relative fluorescence of approximately 1070000. Relative fluorescence for Aβ fibrils at 48 hours with 50 μM SAG was shown to be 580000. Relative fluorescence for Aβ fibrils at 48 hours treatment with 50 μM T-SA was shown to be approximately 940000. Relative fluorescence for Aβ fibrils at 48 hours with 50 μM neoliquiritin was shown to be approximately 600000.

Moving on to the samples that were subjected to 60 hours of treatment with Aβ protein, the sample of only Aβ protein as a starting material had a relative fluorescence of approximately 1010000. Relative fluorescence for Aβ fibrils at 60 hours with 50 μM SAG was shown to be 620000. Relative fluorescence for Aβ fibrils at 60 hours with 50 μM T-SA was shown to be approximately 940000. Relative fluorescence for Aβ fibrils at 60 hours with 50 μM neoliquiritin was shown to be approximately 620000.

Moving on to the samples that were subjected to 72 hours of treatment with Aβ protein, the sample of only Aβ protein as a starting material had a relative fluorescence of approximately 980000. Relative fluorescence for Aβ fibrils at 72 hours treatment with 50 μM SAG was shown to be approximately 590000. Relative fluorescence for Aβ fibrils at 72 hours with 50 μM T-SA was shown to be approximately 770000. Relative fluorescence for Aβ fibrils at 72 hours with 50 μM neoliquiritin was shown to be approximately 640000.

### Dosage

In an exemplary embodiment, a dose composition containing a concentration of about 10 μM of a prodrug configured to deliver SAG may be administered to a patient suffering from cognitive disorders such as Alzheimer's disease. In various embodiments a dose composition containing a concentration of between about 10 μM and 150 μM of a prodrug configured to deliver SAG may be administered to a patient suffering from a cognitive disorder. In various embodiments a dose composition containing a concentration of between about 20 μM and 130 μM of a prodrug configured to deliver SAG may be administered to a patient suffering from a cognitive disorder. In various embodiments a dose composition containing a concentration of between about 30 μM and 110 μM of a prodrug configured to deliver SAG may be administered to a patient suffering from a cognitive disorder. In various embodiments a dose composition containing a concentration of between about 50 μM and 100 μM of a prodrug configured to deliver SAG may be administered to a patient suffering from a cognitive disorder. In various embodiments a dose composition containing a concentration of between about 60 μM and 80 μM of a prodrug configured to deliver SAG may be administered to a patient suffering from a cognitive disorder.

In an exemplary embodiment, a dose composition containing a concentration of about 10 μM of a prodrug configured to deliver neoliquiritin may be administered to a patient suffering from cognitive disorders such as Alzheimer's disease. In various embodiments a dose composition containing a concentration of between about 10 μM and 150 μM of a prodrug configured to deliver neoliquiritin may be administered to a patient suffering from a cognitive disorder. In various embodiments a dose composition containing a concentration of between about 20 μM and 130 μM of a prodrug configured to deliver neoliquiritin may be administered to a patient suffering from a cognitive disorder. In various embodiments a dose composition containing a concentration of between about 30 μM and 110 μM of a prodrug configured to deliver neoliquiritin may be administered to a patient suffering from a cognitive disorder. In various embodiments a dose composition containing a concentration of between about 50 μM and 100 μM of a prodrug configured to deliver neoliquiritin may be administered to a patient suffering from a cognitive disorder. In various embodiments a dose composition containing a concentration of between about 60 μM and 80 μM of a prodrug configured to deliver neoliquiritin may be administered to a patient suffering from a cognitive disorder.

Many variations may be made to the embodiments described herein. For instance, the amounts of the identified prodrugs and various of prodrugs, may comprise different concentrations for an effective dose and treatment. All variations are intended to be included within the scope of this disclosure, including combinations of variations. The description of the embodiments herein can be practiced in many ways. Any terminology used herein should not be construed as restricting the features or aspects of the disclosed subject matter. The scope should instead be construed in accordance with the appended claims.

1. A method for treatment of a patient, the method comprising:
administering an effective dose of a compound having the formula: 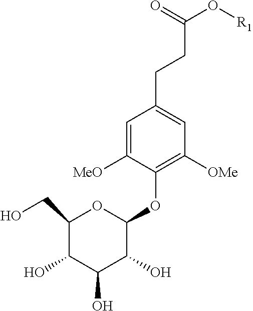
wherein R₁ is selected from the group consisting of: 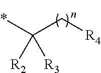
wherein n is an integer selected from 0 to 5, R₂, R₃, and R₄ are independently selected from the group consisting of H, C₁-C₆ alkyl, C₃-C₆ cycloalkyl, C₃-C₆ heterocycloalkyl, Ar, hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr; 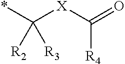
wherein X comprises O, NR, or bond, wherein R₂ and R₃ independently comprises H, C₁₋₆ alkyl, C₃₋₆ cycloalkyl, or cyclized to form a ring, the latter three groups containing or substituted with 0-3 heteroatoms, Ar, or hAr, wherein R₄ is selected from the group consisting of C₁₋₆ alkyl, C₃₋₆ cycloalkyl, the latter two groups containing or substituted with 0-3 heteroatoms, NH-alkyl, N(alkyl)₂, Ar, or hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; 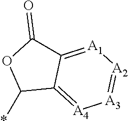
wherein A₁, A₂, A₃, and A₄ are independently selected from the group consisting of N or CR₂, wherein R₂ is selected from the group consisting of H, C₁₋₆ alkyl, C₃₋₆ cycloalkyl, Ar, or hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; 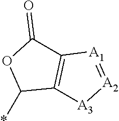
wherein A₁, A₂, and A₃ are independently selected from the group consisting of N, O, S, or CR₂, wherein R₂ is selected from the group consisting of H, C₁₋₆ alkyl, C₃₋₆ cycloalkyl, Ar, or hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; 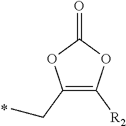
wherein R₂ is selected from the group consisting of H, C₁₋₆ alkyl, C₃₋₆ cycloalkyl comprising or substituted with 0-3 heteroatoms, Ar, or hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; and 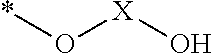
wherein X is selected from the group consisting of C₂₋₆ alkyl, C₃₋₆ cycloalkyl, the latter two groups each containing or substituted with 0-3 heteroatoms, Ar, hAr, or formula I with structure 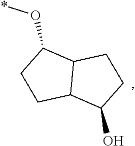 [SMILES: *O[C@H]1CCC2C1CC[C@H]2O]
wherein Ar comprises phenyl independently substituted with 0-5 substituents wherein each substituent is independently selected from C₁-C₆alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)² or a halogen.

2. The method of claim 1, wherein the effective dose has a concentration of between about 10 μM and about 150 μM.

3. The method of claim 2, wherein the effective dose has a concentration of between about 50 μM and about 100 μM.

4. The method of claim 2, wherein the effective dose has a concentration of between about 100 μM and about 150 μM.

5. The method of claim 2, wherein the effective dose has a concentration of between about 20 μM and about 60 μM.

6. The method of claim 2, wherein the effective dose has a concentration of between about 60 μM and about 100 μM.

7. The method of claim 2, wherein the effective dose has a concentration of between about 100 μM and about 140 μM.

8. A method for treatment of a patient, the method comprising:
administering an effective dose of a compound having the formula: 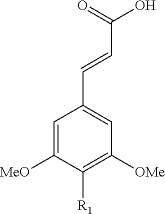
wherein R₁ is selected from the group consisting of: 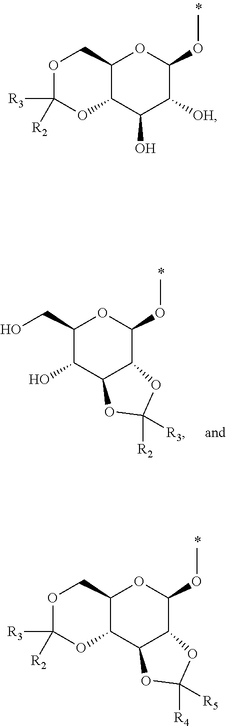
wherein R₂₋₅ are independently selected from the group consisting of H, C₁₋₆ Alk, C₃₋₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen;
or alternatively R₁ is selected from the group consisting of: 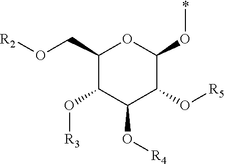
wherein R₂₋₅ are independently selected from the group consisting of H, C₁₋₆ alkyl, C₃₋₆ cycloalkyl, Ar, hAr, or group selected from: 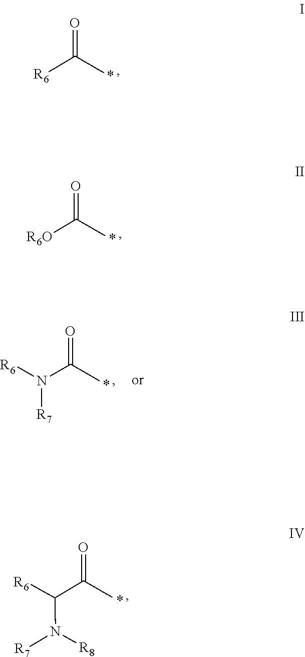
wherein R₂₋₅ are independently selected from the group consisting of H, C₁₋₆ alkyl, C₃₋₆ cycloalkyl, Ar, hAr, I, II, III, or IV;
wherein R₆₋₈ are independently selected from the group consisting of H, C₁₋₆ alkyl, C₃₋₆ cycloalkyl, Ar, or hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising _{C1-6} alkyl containing 0-3 heteroatoms, Ar, or hAr substituted with alkyl, O-alkyl, NH-alkyl, N(alkyl)₂ or a halogen.

9. The method of claim 8, wherein the effective dose has a concentration of between about 10 μM and about 150 μM.

10. The method of claim 9, wherein the effective dose has a concentration of between about 50 μM and about 100 μM.

11. The method of claim 9, wherein the effective dose has a concentration of between about 100 μM and about 150 μM.

12. The method of claim 9, wherein the effective dose has a concentration of between about 20 μM and about 60 μM.

13. The method of claim 9, wherein the effective dose has a concentration of between about 60 μM and about 100 μM.

14. The method of claim 9 wherein the effective dose has a concentration of between about 100 μM and about 140 μM.

15. A method for treatment of a patient, the method comprising:
administering an effective dose of a compound having the formula: 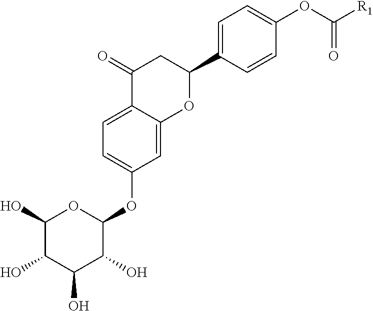
wherein R₁ is selected from the group consisting of: 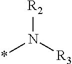
wherein R₂ and R₃ are independently selected from the group consisting of: H, C₁-C₆ alkyl, C₃-C₆ cycloalkyl, or cyclized to form a ring wherein each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; 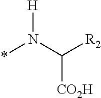
wherein R₂ is independently selected from the group consisting of: H, C₁-C₆ alkyl, C₃-C₆ cycloalkyl, or cyclized to form a ring wherein each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with alkyl, O-alkyl, NH-alkyl, N(alkyl)₂ or a halogen; 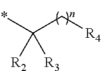
wherein n is an integer selected from 0 to 5, wherein R₂, R₃, and R₄ are independently selected from the group consisting of: H, C₁-C₆ alkyl, C₃-C₆ cycloalkyl, or cyclized to form a ring wherein each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, or hAr, wherein hAr comprises a 5-7 membered heteroaryl ring independently substituted with 0-6 substituents comprising C₁₋₆ alkyl containing 0-3 heteroatoms, Ar, or hAr substituted with hAr substituted with alkyl, O-alkyl, NH-alkyl, N(alkyl)₂ or a halogen; and 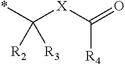
wherein X is selected from the group consisting of O, NR, or a bond, R₂ and R₃ are independently selected from the group consisting of: H, C₁-C₆alkyl, C₃-C₆ cycloalkyl, or cyclized to form a ring wherein each ring contains or is substituted with 0-3 heteroatoms, Ar, hAr, wherein Ar comprises phenyl independently substituted with 0-5 substituents wherein each substituent is independently C₁-C₆ alkyl, C₃-C₆ cycloalkyl, Ar, hAr(alkyl, O-alkyl, NH-alkyl, N(alkyl)₂, or a halogen, wherein hAr comprises a 5-7 membered heteroaryl ring independently substituted with 0-6 substituents comprising C₁₋₆ alkyl containing 0-3 heteroatoms, Ar, hAr substituted with alkyl, O-alkyl, NH-alkyl, N(alkyl)₂ or a halogen.

16. The method of claim 15, wherein the effective dose has a concentration of between about 10 μM and about 150 μM.

17. The method of claim 16, wherein the effective dose has a concentration of between about 50 μM and about 100 μM.

18. The method of claim 16, wherein the effective dose has a concentration of between about 100 μM and about 150 μM.

19. The method of claim 16, wherein the effective dose has a concentration of between about 40 μM and about 80 μM.

20. The method of claim 16, wherein the effective dose has a concentration of between about 80 μM and about 140 μM.
